(19) United States
(12) Patent Application Publication
(10) Pub. No.: US 2019/0376957 A1
(43) Pub. Date: Dec. 12, 2019

(54) SOLID PHASE CONJUGATE

(71) Applicant: BBI Solutions OEM Limited, Cardiff (GB)

(72) Inventors: Ffion Walters, Cardiff (GB); Morgan West, Cardiff (GB)

(21) Appl. No.: 16/331,842

(22) PCT Filed: Sep. 8, 2017
(86) PCT No.: PCT/GB2017/052625

§ 371 Date: Mar. 8, 2019

(30) Foreign Application Priority Data
Sep. 8, 2016 (GB) 1615233.2

(51) Int. Cl. G01N 33/53; C12Q 1/6834

(52) U.S. Cl. CPC G01N 33/5306; C12Q 1/6834

(57) ABSTRACT

The present invention relates to a solid phase biomolecule conjugate for use in a detection assay, comprising on the surface thereof a population of oligonucleotides, and a specific binding partner. The oligonucleotides serve to block the sites which are not bound by a specific binding partner and increase sensitivity for a target molecule to be detected in a sample.

The present invention relates to a solid phase biomolecule conjugate which is adapted to increase sensitivity for a target molecule in a sample. The present invention also relates to a method of making such a solid phase biomolecule conjugate. The present invention also relates to a method of increasing the sensitivity of an analyte detection assay, and to the use of a solid phase biomolecule conjugate of the invention in a method of increasing the sensitivity of an analyte detection assay; and to methods of detection of a target molecule in a sample using the solid phase biomolecule conjugate of the invention. The present invention also relates to a kit comprising a solid phase biomolecule conjugate of the invention.

### BACKGROUND

Solid phases such as arrays and nanoparticles, comprising a binding partner for a target molecule, have been utilised in many diagnostic applications, dating back to the 1980s when conjugates of colloidal gold for recognition of biomolecules began to be used. Today, such conjugates have a wide range of diagnostic applications, such as in ultra-high throughput screening, chip-based technology (e.g. lab on a chip), multi-target detection systems, diagnostic screening and diagnosis. Such conjugates have also found utility in therapeutic applications, for example targeting of therapies to particular sites within a subject.

The utility of such conjugates in diagnostic applications is based upon a fine balance of specific and robust binding of a target molecule to the surface of the solid phase, whilst minimizing non-specific binding of biomolecules to the conjugate. In some aspects, for example where a solid phase is provided in the form of nanoparticles, it is also desirable to minimise aggregation and/or precipitation of the conjugates, particularly in a hydrophobic medium. Increasing sensitivity of a solid phase to a target molecule, reducing non-specific binding of the solid phase to other biomolecules in the sample and reducing aggregation of solid phases are all major limitations in the use of conjugate technology in diagnostic applications.

Typically, agents such as bovine serum albumin (BSA) or PEG have been used to coat a solid phase, to reduce aggregation of solid phase particles and reduce non-specific binding of the solid phase conjugate to biomolecules other than the target molecule which may be present in the sample. However, there are limitations relating to the use of BSA, particularly in diagnostic applications.

The present invention therefore aims to provide an improved solid phase for biomedical and diagnostic applications.

### SUMMARY OF THE INVENTION

In a first aspect of the present invention, there is provided a solid phase-biomolecule conjugate for detection of a target molecule in a sample, the conjugate comprising a solid phase comprising a surface, wherein the surface has bound thereto i) a binding partner specific for the target molecule, and ii) a population of oligonucleotides which do not bind the target molecule, and wherein each oligonucleotide is between about 2 to about 200 nucleotides in length.

The oligonucleotides of the population, by virtue of sequence, structure and/or chemical modification, may not bind to any other molecule present in the sample, either in a specific and/or non-specific manner. The oligonucleotides of the population may therefore be referred to as non-binding. Therefore, in an embodiment of the first aspect, the population of oligonucleotides substantially do not bind to the target molecule, or to any other molecule which may be present in a sample containing or suspected of containing the target molecule. Suitably, the oligonucleotides of the population do not bind specifically to the target molecule or any other molecule which may be present in a sample. In addition, or alternatively, suitably the oligonucleotides of the population do not bind non-specifically to the target molecule or any other molecule which may be present in a sample. In addition, suitably the oligonucleotides of the population do not bind to the specific binding partner.

In an embodiment of the first aspect, the specific binding partner is not a nucleic acid molecule. In such an embodiment, the binding partner may be a protein, polysaccharide, hapten or a non-biological molecule. In an embodiment, the specific binding partner is a protein, for example an antibody, antigen or a receptor, or a fragment thereof. In an embodiment, the solid phase comprises a gold surface, suitably is a gold particle, suitably a gold nanoparticle.

In an embodiment of the first aspect, the population of oligonucleotides are provided on the surface of the solid phase over substantially the entire surface which is not bound by the binding partner. The population may cover the surface of the solid phase which is not bound by the binding partner in a uniform or non-uniform manner.

In a most suitable embodiment, there is provided a gold nanoparticle-biomolecule conjugate for detection of a target molecule in a sample, the gold nanoparticle comprising bound to a surface thereof i) a protein specific for the target molecule, and ii) a population of oligonucleotides which do not bind to the target molecule, and wherein each oligonucleotide is between about 2 to about 200 nucleotides in length. In an embodiment, the protein is an antibody, antigen or a receptor, or a fragment thereof. In an embodiment, the target molecule is not a nucleic acid molecule. In an embodiment, the population of oligonucleotides do not bind to the target molecule which may be present in the sample, or to the specific binding partner. In an embodiment, the population of oligonucleotides do not bind to any other molecule present in the sample. In an embodiment, the population of oligonucleotides are provided on the surface of the solid phase over substantially the entire surface which is not bound by the binding partner. The population may cover the surface of the solid phase which is not bound by the binding partner in a uniform or non-uniform manner.

In an embodiment of the first aspect, the specific binding partner is a nucleic acid molecule. In such an embodiment, the binding partner is specific for the target molecule, which may be a nucleic acid molecule. In a suitable embodiment, the population of oligonucleotides do not bind to the target molecule. In an embodiment, the population of oligonucleotides do not bind to any other molecule present in the sample. Where the target molecule is a nucleic acid molecule, the population of oligonucleotides do not bind to the nucleic acid molecule, a substantially identical sequence, a sequence complementary to the target nucleic acid molecule, an extension product of the target nucleic acid molecule, or a ligation product of the target nucleic acid molecule. Where the specific binding partner is a nucleic acid molecule, the oligonucleotides of the population do not bind to a substantially identical sequence, a sequence complementary to the binding partner nucleic acid molecule, an extension product of the binding partner nucleic acid molecule, or a ligation product of the binding partner nucleic acid molecule.

In an embodiment of the first aspect, the oligonucleotides of the population do not bind to the target molecule or to any other molecule present in the sample by virtue of their sequence or chemical modification. Suitably, the oligonucleotides of the population are not capable of forming a higher-order structure that has specific or non-specific target-binding properties. For example, suitable oligonucleotides of the population may comprise one or more abasic residues which serve to prevent the oligonucleotide from binding, and/or the oligonucleotide may be a double stranded nucleic acid molecule. An example is a 5-12 residue poly A-poly T duplex.

In a second aspect of the present invention, there is provided a method for making a solid-phase biomolecule conjugate for detection of a target molecule in a sample, wherein the method comprises i) providing a solid phase having a surface; ii) attaching a specific binding partner for a target molecule to the surface; and iii) attaching a population of oligonucleotides to the surface, wherein the oligonucleotides of the population do not bind to the target molecule, and wherein each oligonucleotide is between about 2 to about 200 nucleotides in length. The solid-phase biomolecule conjugate may be defined as above in relation to the first aspect.

The conjugates of the present invention may be used in assays for the detection of a specific biomolecule, for example for identifying the presence of a target molecule in a sample and isolating and/or purifying a target molecule. Thus, in a third aspect, the present invention provides for the use of a solid-phase biomolecule conjugate for detection of a target molecule in a sample, wherein the conjugate comprises a solid phase having a surface, wherein the surface has attached thereto i) a specific binding partner for the target molecule, and ii) a population of oligonucleotides which do not bind to the target molecule, and wherein each oligonucleotide is between about 2 to about 200 nucleotides in length. The solid-phase biomolecule conjugate and embodiments thereof may be as defined above in relation to the first aspect.

In a third aspect, the present invention relates to the use of a solid-phase biomolecule conjugate for increasing the sensitivity of an assay for detection of a target molecule in a sample, wherein the conjugate comprises a solid phase having a surface, wherein the surface has attached thereto i) a specific binding partner for the target molecule, and ii) a population of oligonucleotides which do not bind to the target molecule, and wherein each oligonucleotide is between about 2 to about 200 nucleotides in length. The solid-phase biomolecule conjugate may be defined as above in relation to the first aspect.

In a fourth aspect, the present invention provides a method of detecting a target molecule, the method comprising

a) providing a solid-phase biomolecule conjugate, wherein the conjugate comprises a solid phase having a surface, wherein the surface has attached thereto i) a specific binding partner for the target molecule, and ii) a population of oligonucleotides which do not bind to the target molecule, and wherein each oligonucleotide is between about 2 to about 200 nucleotides in length;

b) contacting the solid-phase biomolecule conjugate with a sample suspected of containing the target molecule;

c) incubating the sample and the solid phase biomolecule conjugate for sufficient time to allow specific binding of the conjugate to any target molecule present in the sample; and

d) detecting a change which occurs upon binding of any target molecule to the conjugate.

A method of the invention may be used to isolate and/or purify a target molecule present in a sample. The methods and uses of the invention also relate to increasing the sensitivity of an assay for detection of a target molecule in a sample, by the use of a conjugate.

In the methods and uses of the third and fourth aspects of the invention, the solid-phase biomolecule conjugate may be as defined above in relation to the first aspect.

Therefore, the oligonucleotides of the population, by virtue of sequence, structure and/or chemical modification, may not bind to any other molecule present in the sample, either in a specific and/or non-specific manner. The oligonucleotides of the population may therefore be referred to as non-binding. Therefore, in an embodiment of the third and fourth aspects, the population of oligonucleotides substantially do not bind to the target molecule, or to any other molecule which may be present in a sample containing or suspected of containing the target molecule. Suitably, the oligonucleotides of the population do not bind specifically to the target molecule or any other molecule which may be present in a sample. In addition, or alternatively, suitably the oligonucleotides of the population do not bind non-specifically to the target molecule or any other molecule which may be present in a sample. In addition, suitably the oligonucleotides of the population do not bind to the specific binding partner.

In an embodiment of the third and fourth aspects, the specific binding partner is not a nucleic acid molecule. The binding partner may be a protein, polysaccharide, hapten or a non-biological molecule. In an embodiment, the specific binding partner is a protein, for example an antibody, antigen or a receptor, or a fragment thereof. In an embodiment, the solid phase comprises a gold surface, suitably is a gold particle, suitably a gold nanoparticle.

In an embodiment of the third and fourth aspects, the population of oligonucleotides are provided on the surface of the solid phase over substantially the entire surface which is not bound by the binding partner. The population may cover the surface of the solid phase which is not bound by the binding partner in a uniform or non-uniform manner.

In a most suitable embodiment, there is provided a use or method as defined in the third or fourth aspect, wherein the solid phase biomolecule conjugate is a gold nanoparticle-biomolecule conjugate comprising bound to a surface thereof i) a protein specific for the target molecule, and ii) a population of oligonucleotides which do not bind to the target molecule, and wherein each oligonucleotide is between about 2 to about 200 nucleotides in length. In an embodiment, the protein is an antibody, antigen or a receptor, or a fragment thereof. In an embodiment, the target molecule is not a nucleic acid molecule. In an embodiment, the population of oligonucleotides do not bind to the target molecule which may be present in the sample. In an embodiment, the population of oligonucleotides do not bind to any other molecule present in the sample. In an embodiment, the population of oligonucleotides are provided on the surface of the solid phase over substantially the entire surface which is not bound by the binding partner. The population may cover the surface of the solid phase which is not bound by the binding partner in a uniform or non-uniform manner.

In an embodiment of the third and fourth aspects, the specific binding partner is a nucleic acid molecule. In such an embodiment, the binding partner is specific for the target molecule, which may be a nucleic acid molecule. In a suitable embodiment, the population of oligonucleotides do not bind to the target molecule. In an embodiment, the population of oligonucleotides do not bind to any other molecule present in the sample. Where the target molecule is a nucleic acid molecule, the population of oligonucleotides do not bind to the nucleic acid molecule, a substantially identical sequence, a sequence complementary to the target nucleic acid molecule, an extension product of the target nucleic acid molecule, or a ligation product of the target nucleic acid molecule. Where the specific binding partner is a nucleic acid molecule, the oligonucleotides of the population do not bind to a substantially identical sequence, a sequence complementary to the binding partner nucleic acid molecule, an extension product of the binding partner nucleic acid molecule, or a ligation product of the binding partner nucleic acid molecule.

In an embodiment of the third and fourth aspects, the oligonucleotides of the population do not bind to the target molecule or to any other molecule present in the sample by virtue of their sequence or chemical modification. Suitably, the oligonucleotides of the population are not capable of forming a higher-order structure that has specific or non-specific target-binding properties. For example, suitable oligonucleotides of the population may comprise one or more abasic residues which serve to prevent the oligonucleotide from binding, and/or the oligonucleotide may be a double stranded nucleic acid molecule. An example is a 5-12 residue poly A-poly T duplex.

### FIGURES

The present invention is described with reference to the drawings, in which:

FIG. 1 shows half dipsticks at completion of a detection assay using a conjugate of the invention.

FIG. 2 shows the mean signal intensities demonstrating the comparison between oligonucleotide blocked and BSA blocked anti-BNP conjugates.

FIG. 3 shows a comparison of the mean Camag reader, test-line signals produced during the half dipstick testing using unhybridized polyT oligonucleotides and hybridised polyA polyT oligonucleotides as blocking agents.

FIG. 4 are photographs of 100 IU/L hCG concentration strips (A) and 10 IU/L hCG concentration strips (B) where n=5 strips, poly T unhybridised (control) conjugate strips are shown on the left, and poly T—poly A hybridised conjugate strips on the right.

FIG. 5 are photographs of 1 IU/L hCG concentration strips (A) and negative sample strips (B) where n=5 strips, poly T unhybridised (control) conjugate strips on left, poly T—poly A hybridised conjugate strips on right.

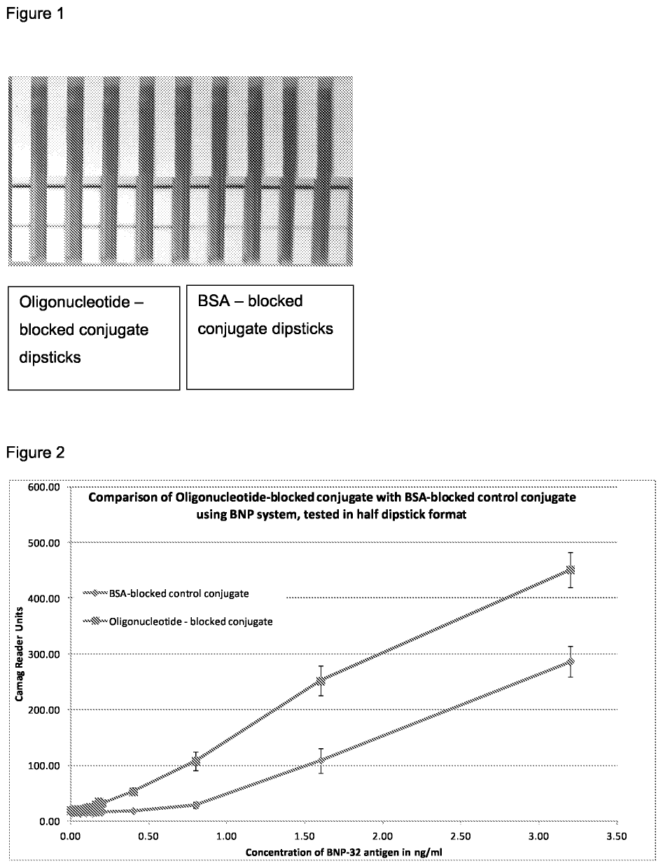

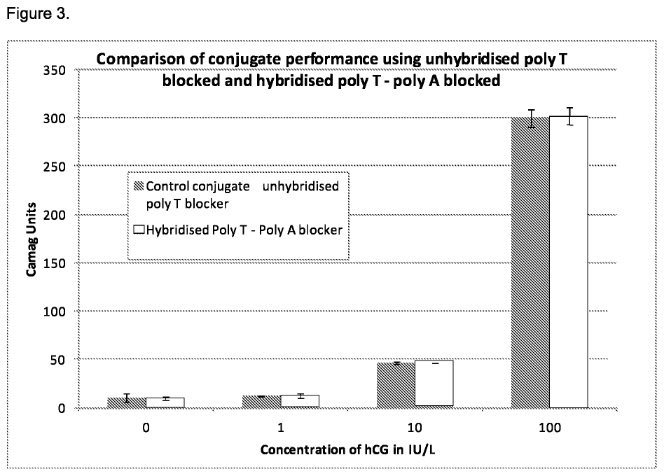

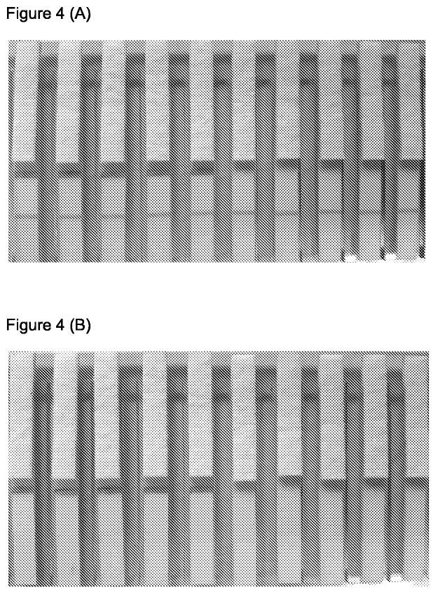

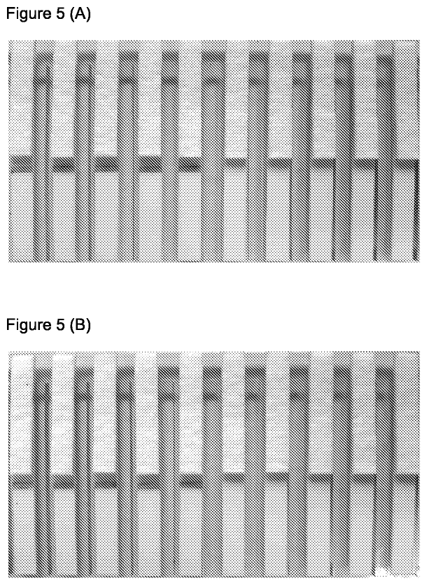

### DETAILED DESCRIPTION

The present invention is based upon the surprising discovery that using a population of oligonucleotides to block the sites of a solid phase which are not bound by a specific binding partner can increase sensitivity of a specific binding partner for a target molecule, compared to conventional blocking agents such as BSA. The present inventors have observed that a population of small, non-specific oligonucleotides which are non-binding (such that they do not bind to the target molecule to be detected in a sample, and preferably substantially do not bind any other molecule or the specific binding partner) can be used as a blocking agent. Due at least in part to their reduced size compared to conventional blocking agents such as BSA or PEG, the oligonucleotide population of the solid phase conjugate serves to increase sensitivity of the detection assay compared to sensitivity when blocking agents such as BSA are used. In addition, a population of oligonucleotides as defined herein serves to reduce or minimise self-aggregation of the solid phase conjugates and non-specific binding of the conjugates to other molecules in the sample. Selectivity of the solid phase for a target molecule is also increased compared to a solid phase coated with a conventional blocking agent such as BSA. Where the solid phase is a nanoparticle, the provision of a population of oligonucleotides as described herein on the surface of the solid phase, can also serve to reduce non-specific aggregation of the particles.

The invention has the further advantage that any cross reaction, for example between BSA and bovine material, in veterinary assays is avoided.

Therefore, the provision of a population of oligonucleotides as described herein, on the surface of the solid phase in addition to the specific binding partner for the target molecule serves to increase assay sensitivity for the target molecule. The oligonucleotide is designed not to interfere with or inhibit binding of the target molecule to the specific binding partner. The population may also serve to inhibit non-specific binding of biomolecules to the surface of the solid phase. In an embodiment, the population of oligonucleotides substantially coat the entire surface of the solid phase which is not bound by the specific binding partner.

### Solid Phase

A solid phase may be any suitable size and shape, such as a chip, a particle (for example a nanoparticle), a well, a cuvette, a column, an array, a dipstick, a membrane, quantum dots or a bead. In some embodiments, the solid phase is a rectangular chip or a disc, or the bottom, the cover, and/or interior walls of a well or cuvette (e.g., cylindrical or rectangular cuvette).

A solid phase comprises a surface to which the specific binding partner and the oligonucleotide of the invention are attached either directly or indirectly (e.g. via linkers or functional groups). The surface may be modified or functionalised in any suitable way to mediate attachment of the specific binding partner and/or oligonucleotide. Binding sites (modified, functionalised or native surface) are the points or areas of the surface to which attachment occurs, and may be referred herein to as active sites or attachment sites. The surface may be suitable for attachment by covalent or non-covalent bonds.

A solid phase may be any material suitable for applications of the present invention, such as diagnostic applications of detecting a target molecule in a sample.

A solid phase may be any suitable material, including metallic or non-metal material, and may include gel-like or semi-solid materials. A solid phase may comprise two or more materials. The surface may comprise one or more different materials to the remainder of the solid phase.

Metallic materials include gold, silver, platinum, aluminium, palladium, copper, cobalt, indium, titanium, iron oxide, zinc, nickel, or mixtures thereof. Therefore, a solid phase or a surface thereof may be pure metal or may be a combination or a mixture, for example an alloy of two or more suitable metals. Non-metallic materials suitable for use as a solid phase include without limitation, silica, latex, polystyrene, plastic, cellulose (for example nitrocellulose), and carbon. A solid phase or a surface thereof may be a pure non-metallic material or a mixture or combination of two or more non-metallic materials. Also envisaged for use in the present invention is a solid phase or a surface thereof comprising a mixture or combination of metallic and non-metallic materials. In such an embodiment, the surface may be a single material or a mixture or combination of metallic and non-metallic materials. In the case of particles, for example, a core of one material may be provided, and an outer shell comprising the surface may be provided, wherein the outer shell comprises a second or further material, or a mixture or combination of two or more materials.

Herein, reference to a pure material means that it is substantially composed of a single material, for example it comprises at least 90% or more of a single material. A mixture refers to two or more materials which have been physically but not necessarily chemically combined. An alloy is an example of a mixture. A combination refers to two or more separate materials, of the solid phase or surface.

In an embodiment, the solid phase is a particle, preferably a nanoparticle. A particle may be spherical or rod-shaped. Where spherical, size of a nanoparticle is preferably from about 1 nm to about 1 μm in diameter, more suitably from about 5 nm to about 500 nm, more suitably from about 5 nm to about 200 nm, most suitably from about 10 to about 150 nm, most suitably 20 to 50, or 40 nm in diameter. Where rod-shaped, a nanoparticle may possess one or more dimensions between 1 nm and 1 μm, more suitably from about 5 nm in width to about 25 nm in width, most suitably from about 25 nm in length to about 250 nm in length. Where rod shaped, a nanoparticle may have an aspect ratio of one or more (length to width). A nanoparticle may be anisotropic or isotropic.

The size of the nanoparticles may vary depending on their proposed use or application. The variation of size may be used to optimize certain physical characteristics of the nanoparticles, for example, optical properties or amount of surface area that can be derivatized as described herein.

Particularly suitable for use in present invention are gold surfaces, in particular gold nanoparticles comprising a gold surface. Substantially all of the particle may be gold, or substantially all of the surface thereof may be gold. In an embodiment, a core other than gold may be provided. In an embodiment, a particle may be hollow and may comprise a shell having a surface for attachment. In such an embodiment, the shell and surface may have the same or different material(s). Gold nanoparticles are particularly suited for colorimetric assays and also because of their stability, ease of imaging by electron microscopy, and well-characterized modification with thiol functionalities. In an embodiment, spherical gold nanoparticles having a diameter of 30-50 nm, more suitably 40 nm, are used.

In an embodiment, a composite metallic comprises gold and silver, gold and copper, or silver and copper. In some embodiments, a core comprising a first metal is dissolved with a second metal to create a hollow structure comprised of the second metal. For instance, coating of a silver core with gold nanoparticles generates a gold shell around the silver core and the silver core is subsequently dissolved or degraded resulting in the formation of a hollow gold shell structure.

A particle may be monodisperse (a single crystal) or polydisperse (comprising a plurality of crystals).

Where a population of solid phases are used in any particular application, these may comprise a homogenous population comprising a single type of solid phase, or may comprise a mixture of two or more different types of solid phase. For example, where the solid phase is particulate, a mixture may comprise spherical and rod-shaped particles, or particles of different materials, or bound by different specific binding particles and/or different blocking oligonucleotides. Such a population may be referred to herein as a heterogenous population.

### Specific Binding Partner

A specific binding partner provided on the surface of the solid phase may be any molecule which preferentially binds to a target molecule, either covalently or non-covalently, for use in detection. A specific binding partner may be a biological molecule, for example a nucleic acid molecule, a polypeptide (e.g. a protein or peptide) such as a receptor or antibody or fragment thereof, a hapten or a polysaccharide. Non-biological specific binding partners include, for example, small molecules and drugs such as MIPs. A specific binding partner may be other than a nucleic acid molecule. Where the specific binding partner is a nucleic acid molecule it may include for example, DNA, RNA, cDNA, siRNA, miRNA, and snRNA. A specific binding partner provided as a nucleic acid molecule may be single stranded or double stranded, preferably the former. A nucleic acid molecule for use as a specific binding partner may be antisense to a nucleic acid target molecule to be detected. It may be synthetic or natural. A nucleic acid for use as a specific binding partner may be an aptamer.

A specific binding partner may be a polypeptide, such as an immunoglobulin binding moiety, an antigen, receptor, ligand or hormone, lipoprotein or a nucleoprotein. An immunoglobulin binding moiety may be an antibody or antibody fragment. The term “full-length antibody” refers to a protein that includes one polypeptide that includes a light chain and one heavy chain. The term “antibody fragment” refers to one or more fragments of a full-length antibody that retain the ability to specifically bind to a target. Examples of an antibody fragment include, but are not limited to, a Fab fragment, a F(ab′)₂ fragment, an Fd fragment, an Fv fragment, a dAb fragment, a CDR and an scFv. An antibody can be monoclonal or polyclonal and can be prepared by techniques that are well known in the art. An antibody or fragment thereof may be of any suitable class and isotype, such as IgA, IgD, IgE, IgGI, IgG2a, IgG2b and IgG3, IgM, etc. In addition, aggregates, polymers, and conjugates of immunoglobulins or their fragments can be used where appropriate so long as binding affinity for a target molecule is maintained.

A specific binding partner may be a saccharide, for example mono-, di-, tri- and polysaccharides for example glycosides, lipopolysaccharide, N-glycosylamines, O-acyl derivatives, O-methyl derivatives, osazones, sugar alcohols, sugar acids, and sugar phosphates. A polysaccharide can be synthetic or natural.

Generally herein, by specific binding is meant that a molecule, such as a specific binding partner, binds preferentially to another molecule, such as the target molecule, in the presence of other molecules. Herein, specific binding refers to preferential binding, suitably of high affinity. By non-specific binding is meant binding of a molecule to something other than its designated target, preferably with low affinity. Herein, reference to non-specific binding may include the binding of a variety of biomolecules present in a sample to the surface of solid phase and not to a designated target such as a specific binding partner.

In an embodiment, one or more specific binding partners are provided on the solid phase. One or more may be referred to as a population, of any suitable size. The specific binding partners may evenly or unevenly coat the surface of a solid phase. The specific binding partners may densely cover the surface of a solid phase, substantially cover the surface of a solid phase, or sparsely cover the surface of a solid phase. A higher density of specific binding partners may increase the detection of a target molecule in a sample. Preferably, the specific binding partners is provided at any suitable density, which may depend upon factors such as the application of the solid phase, the target molecule, the desired sensitivity of the assay, the sample type and the specific binding partner. A suitable density may be determined using methods known in the art by a skilled person.

Where two or more specific binding partners are provided, they may be the same or different. They may be specific for the same or different target molecules. A population comprising two or more different specific binding partners may be referred to as a heterogeneous population. A population comprising a single type of specific binding partner may be referred to as a homogeneous population. Reference herein to a specific binding partner includes a population of specific binding partners. The size of the population will depend in part upon the size of the solid phase. Any population size is included within the scope of the invention.

Methods of conjugating a specific binding partner to a solid surface are described below.

In an embodiment, the solid phase comprises a gold surface, and suitably is a gold particle such as a gold nanoparticle.

### Target Molecule

A target molecule is a molecule to be detected in a sample. A target molecule may be the same type of molecule as defined in relation to the specific binding partner. It may be a biological or non-biological molecule, the detection of which is desired. A target molecule can be any biological molecule, including for example a nucleic acid molecule, a polypeptide (e.g. a protein or peptide), a hapten, or a polysaccharide, or combinations thereof such as nucleic acid-protein complexes, microorganisms, and viruses. A target molecule may be non-biological, for example a small molecule or drug, or a metal ion.

A target nucleic acid molecule may include for example, DNA, RNA, cDNA, siRNA, miRNA, and snRNA. A nucleic acid molecule may be single stranded or double stranded. A target nucleic acid molecule may be genomic, or artificial. It may be bacterial, viral, plant or animal derived. A target nucleic acid may be synthetic or natural.

A target polypeptide may include an immunoglobulin binding moiety, an enzyme, an antigen, a structural protein (e.g. a cell surface protein or an extracellular matrix protein), a hormone (e.g. a cytokine or a growth factor), a receptor, a ligand, a lipoprotein or nucleoprotein. A target polypeptide may have catalytic, signalling, therapeutic, or transport activity. A target polypeptide may be a bacterial, viral, fungal, plant or animal protein. A target polypeptide may be synthetic or natural.

Examples of immunoglobulin binding moieties are antibodies or antibody fragments. A typical antibody comprises a light chain and a heavy chain. The term “antibody fragment” refers to one or more fragments of a full-length antibody that retain the ability to specifically bind to a target. Examples of an antibody fragment include, but are not limited to, a Fab fragment, a F(ab′)₂ fragment, an Fd fragment, an Fv fragment, a dAb fragment, a CDR and an scFv. The antibody can be monoclonal or polyclonal and can be prepared by techniques that are well known in the art. An antibody or antibody fragment thereof may be of any classes and isotypes, such as IgA, IgD, IgE, IgGI, IgG2a, IgG2b and IgG3, IgM, etc. In addition, aggregates, polymers, and conjugates of immunoglobulins or their fragments can be used where appropriate so long as binding affinity for a target molecule is maintained.

Examples of protein hormones include for example platelet-derived growth factor (PDGF); insulin-like growth factor-I and -II (Igf); nerve growth factor (NGF); fibroblast growth factor (FGF, e.g. aFGF and bFGF); epidermal growth factor (EGF); transforming growth factor (TGF, e.g., TGF-α and TGF-β); erythropoietin; growth hormone (e.g., human growth hormone); and proinsulin, insulin, A-chain insulin, and B-chain insulin.

Examples of other target polypeptides include, for example, blood serum protein.

A target polysaccharide may include, for example mono-, di-, tri- and polysaccharides for example glycosides, lipopolysaccharides, N-glycosylamines, O-acyl derivatives, O-methyl derivatives, osazones, sugar alcohols, sugar acids, and sugar phosphates. A polysaccharide can be synthetic or natural. Preferably a target polysaccharide is antigenic.

A target microorganism may include bacteria, virus particles, yeast and fungi.

A target molecule will generally be found in a sample, or is suspected of being present in a sample.

By “present in a sample” with reference to a molecule, may mean that the molecule is available for binding within the sample. Therefore, reference to a molecule (target molecule or other molecule) present in a sample may exclude molecules sequestered within intact organelles or which are otherwise not available for binding to a solid phase biomolecule conjugate applied to the sample. Therefore, present in sample may mean circulating or free molecules, or non-cellular molecules.

### Sample

The term “sample,” or “test sample” as used herein, refers to a composition that is obtained or derived from a subject of interest that is suspected of containing a target molecule that is to be detected, characterized and/or isolated, for example based on physical, biochemical, chemical and/or physiological characteristics.

A sample may be a body fluid or tissue, or an environmental or food source. A body fluid or tissue can be, for example, urine, blood or blood constituents, plasma, serum, saliva, semen, stool, sputum, cerebral spinal fluid, tears, lymph fluid, mucus, seminal fluid, amniotic fluid, milk, whole blood, sputum, perspiration, interstitial fluid, vaginal discharge and the like. Also included are primary or cultured cells or cell lines, cell supernatants, cell lysates, body tissue includes biopsy specimen, tumor lysates, and tissue culture medium, tissue extracts such as homogenized tissue, tumor tissue, cellular extracts, and combinations thereof. In one embodiment, the sample is a clinical sample. Tissue biopsy is often used to obtain a representative piece of tumor tissue. A sample may be examined directly for the target molecule, for example as described herein, or may be pre-treated for example by treatment with reagents, solubilization, or enrichment for certain components, such as proteins or polynucleotides. A method of the invention may therefore comprise a step of pre-treating a sample. The source of a tissue sample may be solid tissue or from a fresh, frozen and/or preserved organ or tissue sample or biopsy or aspirate.

In an embodiment, where a sample comprises free nucleic acid which is available for binding (for example a lysate or nuclear extract), the oligonucleotides of the solid phase conjugate will substantially not bind to any such free nucleic acid, by virtue of its sequence, chemical structure or chemical modification. Suitably, in such an embodiment, the oligonucleotides may be chemically modified such that they are incapable of binding to another nucleic acid molecule, or may be double stranded oligonucleotides. For example, suitable oligonucleotides of the population may comprise one or more abasic residues which serve to prevent the oligonucleotide from binding, and/or the oligonucleotide may be a double stranded nucleic acid molecule. An example is a 5-12 residue poly A-poly T duplex.

In an embodiment where a sample does not substantially comprise circulating or free cellular extracts (e.g. urine or samples processed to remove free or circulating nucleic acids), the oligonucleotides of the solid phase conjugate may accordingly be such that they do not bind to any molecule available for binding in the sample. For use with such a sample, the oligonucleotides may be chemically modified such that they are incapable of binding to another molecule, and may be single stranded or double oligonucleotides.

### Oligonucleotide

The population of oligonucleotides may be referred to herein as a blocking agent.

The population of oligonucleotides, in combination with the specific-binding partner on a solid support, may increase sensitivity of an assay by a variety of mechanisms, operating either singularly or in combination, spatially or temporally. The modes of action may include reducing the non-specific binding of other molecules to the conjugate, improving the availability or presentation of the specific-binding partner to the target, aiding in co-operative binding of several specific-binding partners to the target, increasing the availability of the conjugate in its totality for the assay reaction or structure, or improving the kinetics of the reaction in a rate limiting environment.

The oligonucleotides of the population are suitably designed such that they do not exhibit specific binding, or more suitably any binding with the target molecule. Suitably, the oligonucleotides of the population are designed not to bind specifically to the target molecule or any other molecule present in the sample. Suitably, the oligonucleotides of the population are designed not to exhibit non-specific binding for the target molecule or any other molecule present in the sample. Suitably the oligonucleotides do not exhibit any substantial binding for other oligonucleotides in the population. The oligonucleotides may have a sequence, or a chemical modification, which prevents or significantly reduces their ability to bind to nucleic acid molecules present in the sample. In addition, suitably the oligonucleotides of the population do not bind to the specific binding partner.

By “does not bind”, non-binding or no substantial binding may mean any binding above a minimal degree of binding, such as 0.25, 0.5, 1, 2, 3, 4 or 5% of the oligonucleotide population binding a molecule. Binding may be specific and/or non-specific. “Does not bind” refers to any binding relationship between an oligonucleotide and molecule, either when present on the surface of the conjugate and preferably also when not conjugated to the surface of the solid phase.

Reference to “any other molecule” may be as defined in relation to the target molecule. Therefore, any other molecule to which the oligonucleotide population does not bind may be any biological molecule, including for example a nucleic acid molecule, a polypeptide (e.g. a protein or peptide), a hapten, or a polysaccharide, or combinations thereof such as nucleic acid-protein complexes, microorganisms, and viruses. A target molecule may be non-biological, for example a small molecule or drug, or a metal ion. Suitably, the oligonucleotide population does not exhibit any substantial binding to any of these molecules. Specific example of such molecules are provided above, and are non-exhaustive.

In an embodiment, where the target molecule is a nucleic acid molecule, the oligonucleotides of the population do not bind to the nucleic acid molecule, a substantially identical sequence, a sequence complementary to the target nucleic acid molecule, an extension product of the target nucleic acid molecule, or a ligation product of the target nucleic acid molecule. Suitably, the population of oligonucleotides do not bind any other molecule present in the sample, and suitably the oligonucleotides of the population do not bind to a substantially identical sequence, a sequence complementary to the binding partner nucleic acid molecule, an extension product of the binding partner nucleic acid molecule, or a ligation product of the binding partner nucleic acid molecule.

Suitably, the oligonucleotides of the population do not bind to any other molecular assay component, or derivative thereof. A derivative of a molecular assay component may be any product derived for said component, such as a fragment, complement, ligation product, extension product, conjugates, or compounds.

The population of oligonucleotides may be the sole biomolecule on the solid-support surface in addition to the specific binding partner. In an embodiment, a blocking agent such as BSA is not present on the surface of the solid-phase biomolecule conjugate.

In an embodiment, the oligonucleotides of the population are provided on the surface of the solid phase over substantially the entire surface which is not bound by the binding partner. The population may cover the surface of the solid phase which is not bound by the binding partner in a uniform or non-uniform manner. By uniform means evenly spread. By non-uniform means unevenly spread.

Suitable oligonucleotides for provision in a population are generally from about 2 nucleotides to about 200 nucleotides in length. In general, a longer oligonucleotide may inhibit or interfere with binding of a target molecule to the specific binding partner, and shorter oligonucleotides may be preferred. In an embodiment, an oligonucleotide of a population may independently be about 2 to about 90 nucleotides in length, about 2 to about 80 nucleotides in length, about 2 to about 70 nucleotides in length, about 2 to about 60 nucleotides in length, about 2 to about 50 nucleotides in length about 2 to about 45 nucleotides in length, about 2 to about 40 nucleotides in length, about 2 to about 35 nucleotides in length, about 2 to about 30 nucleotides in length, about 2 to about 25 nucleotides in length, about 5 to about 20 nucleotides in length, about 5 to about 15 nucleotides in length, about 5 to about 10 nucleotides in length, about 8 to 12 nucleotides in length, about 10 to 30 nucleotides in length, and all oligonucleotides intermediate in length of the sizes specifically disclosed to the extent that an oligonucleotide is able to achieve the desired result. Accordingly, oligonucleotides of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 120, 130, 140, 150, 160, 170, 170, 180, 190 or more nucleotides in length are contemplated. Specifically contemplated herein are oligonucleotides having 2 to 200 nucleotides, or 5 to 30 nucleotides, or 5 to 15 nucleotides. Most preferred is an oligonucleotide having 5 to 12 nucleotides.

In an embodiment, the non-binding nature of the oligonucleotides of the population may be a virtue of the sequence. The sequence of the oligonucleotide may be designed to minimise binding thereto, therefore reducing non-specific binding to the solid phase. The oligonucleotides of population do not bind to the target molecule or another molecule in the sample, either specifically or non specifically.

Any suitable sequence may be used, and may be designed with the target molecule in mind (for example to avoid interference with specific binding of the target molecule). The oligonucleotides of the population may each independently comprise a sequence selected from a poly(A), poly(T), poly(C) or poly(G) sequence and/or each independently consist of a sequence selected from a poly(A), poly(T), poly(C) or poly(G) sequence. In an embodiment, a suitable oligonucleotide sequence comprises or consists of a poly(A) and/or poly(T) sequence. A suitable oligonucleotide sequence may alternatively be randomly generated.

In an embodiment, a suitable sequence is 5 to 12 nucleotides and consists of a poly(A) and/or poly(T) sequence. In an embodiment, the oligonucleotide is a 10 base poly(T) sequence with thiol modification (5′ SH-TTT-TTT-TTT-T 3′) (Eurogentec).

In an embodiment, the non-binding nature of the oligonucleotides of the population may be a virtue of chemical modification, for example the addition of a blocking group such as a chemical species. A suitable chemical species may include an abasic nucleotide. One or more abasic nucleotides may be incorporated into an oligonucleotide, at a 5′ or 3′ end, and/or within the sequence. For example, a single abasic nucleotide may be introduced within the oligonucleotide sequence, or basic nucleotides may be provided at suitable intervals (e.g. alternate, or every 2, 3, or more nucleotides), in a regular or irregular pattern.

In an embodiment, the non-binding nature of the oligonucleotides of the population may be a virtue of structure, for example they may be provided as chemical modification duplexes (double stranded oligonucleotide) rather than single stranded oligonucleotides (for example a polyA-polyT duplex), which do not bind to other nucleic acid molecules.

In an embodiment, oligonucleotides of a population may comprise any suitable combination of features of sequence, structure and chemical modification. For example, a suitable oligonucleotide may comprise a poly(A) or poly(T) sequence, and may also comprise chemical modification for example in the form of one or more abasic residues provided in the sequence, and/or may be a double stranded oligonucleotide.

In an embodiment, an oligonucleotide is a 5 to 12 base pair poly(A)-poly(T) duplex.

Oligonucleotides of the population may be thiol-modified.

A population of oligonucleotides comprises two or more oligonucleotides. Preferably, a population comprises a sufficient number of oligonucleotides to substantially coat a surface of a solid phase sufficiently to minimise aggregation of solid phases. A population of oligonucleotides may evenly or unevenly coat the surface of a solid phase. A population may densely, substantially or sparsely cover the surface of a solid phase. A higher density of oligonucleotides may be preferred, to minimise aggregation. The oligonucleotide members of a population may be the same or different, in terms of length, sequence, functional groups, modifications, and/or mode of attachment. A population comprising two or more different oligonucleotides (in terms of length, sequence, functional groups, modifications, and/or mode of attachment) may be referred to as a heterogenous population. A population comprising the same type (in terms of length, sequence, functional groups, modifications, and/or mode of attachment) is referred to as a homogenous population. Preferably, the mode of attachment may be the same for all members of the population. The members of a population may vary in length and/or sequence.

Reference to oligonucleotides of a population may means each oligonucleotide, independently. Oligonucleotides of a population suitable includes substantially all of the oligonucleotides of a population, suitably 80%, 85%, 90%, 95%, 96%, 97%, 98%, 99% or more.

In an embodiment, the length and/or sequence of the oligonucleotides will be selected depending upon the nature of the specific binding partner.

The proportion of specific binding partners:oligonucleotides on a solid phase will preferably be designed to maximise availability of the specific binding for binding to a target molecule.

### Conjugation

Herein, the specific binding partner and oligonucleotide population are independently conjugated to the solid phase surface, either covalently or non-covalently. The oligonucleotide population may be conjugated to the solid phase before, together or after conjugation of the specific binding partner to the solid phase.

The same or different techniques may be used for conjugation of the specific binding partner and the oligonucleotides. By conjugation means attached or bound by covalent or non-covalent means. Non covalent bonds include, for example, van der waals forces.

Methods of conjugating molecules to metallic surfaces are known to those of skill in the art. Such methods include conjugation chemistries, such as those involving I-Ethyl-3-[3-dimethylaminopropyl]carbodiimide hydrochloride (EDC), sulfo-NHS coupling, hydrophobic binding or thioether chemistry. In some embodiments, the molecule can be coupled to the metallic surface indirectly through a larger carrier molecule or protein. Such indirect coupling is particularly useful when the molecule is small, such as a hormone, a drug, and other small molecules less than 10 kD. Preferably, the carrier protein is not capable of specific interaction with the target molecule.

Methods of conjugating molecules to non-metallic surfaces are known to those of skill in the art.

Any suitable amount of a solid phase may be used as a starting material for conjugation. Suitable amounts may be determined based upon the optical density of the solid phase in a liquid phase, for example water. A suitable concentration may be a solution of a solid phase in water having an optical density (OD) of 0.75-1.25, more suitably about 1. Optical density may be measured at A_{520 nm} using any suitable means such as a Hitachi UV/VIS spectrophotometer, model U2800A.

Conjugation of a biological molecule (a specific binding partner and/or oligonucleotide) to the solid surface may be achieved via functional groups, for example associated with the solid surface. In an embodiment, a solid surface may be coated with a polymer which includes the functional groups for attachment to the biological moieties. For example, suitable polymers may include synthetic or natural polymers, such as polyethylene glycol or silane, and combinations thereof. Reference herein to a surface includes a surface coated or modified for attachment. Alternatively, methods are known in the art for attaching molecules to solid surfaces without the need for functional groups.

Incorporation of reactive organic functional groups, particularly primary amine, thiol (sulfhydryl), or carboxylate groups, at specific sites within an oligonucleotide allows for subsequent conjugation of the oligonucleotide to a solid phase surface, such as gold. Such reactive groups may be introduced at the 3′ or 5′ end of a nucleic acid molecule, or at any other position in the nucleic acid molecule. A suitable thiol group may be obtained from dithiol Phosphoramidite (DTPA). Other suitable modifiers used for conjugation are amino and carboxyl.

In an embodiment, the oligonucleotides are conjugated to the solid phase by a covalent linkage, via a functional group introduced into an oligonucleotide. Preferably the functional group is a thiol group. Preferably, the thiol functional group is provided at the 5′ end of the oligonucleotide molecule. Preferably, the solid phase comprises a gold surface. In an embodiment, the solid phase is a nanoparticle comprising a gold surface. Preferably the covalent bond is an Au—S bond formed between the sulphur of the thiol group and the gold surface.

In an embodiment, the covalent bond is formed during an incubation of the solid surface with the oligonucleotide at a final salt concentration of 90-110 mM. In an embodiment, the covalent bond is formed during an incubation of the solid surface with an oligonucleotide at a temperature of above 20° C., preferably between 30° C. and 60° C., preferably 45 to 55° C. The incubation may last at least 30 minutes, preferably at least 45 minutes, preferably at least one hour. It may preferably last less than two hours. It may preferably last between about 1 and 2 hours. In an embodiment, the incubation lasts about 1.5 hours.

A preferred method for conjugation of an oligonucleotides to a gold solid surface is described in “Rapid Synthesis of stable and Functional Conjugates of DNA/Gold Nanoparticles Mediated by Tween 80” Shengmin Xu et al. Langmuir 2011, 27, 13629-13634.

In an embodiment, suitably for gold nanaoparticles, a 10% (v/v) solution of Tween 80 is added to an OD1 antibody-gold nanoparticle mixture, followed by incubation at room temperature, typically 18-24° C., with constant mixing at low speed for 30 minutes. Following the incubation, phosphate buffered saline buffer (PBS) (0.1M Na₂ HPO₄, 0.03M KH₂O₄ P, 1.23M NaCl, pH7.4) is combined with the antibody-gold nanoparticle mixture, preferably dropwise, and more preferably with inversion after each drop. Following incubation with PBS, the oligonucleotide is added to the antibody-gold mixture.

A suitable method may include non covalent attachment by incubation at a pH range of 6.0-9.0 depending on the antibody used.

Conjugation of other biological molecules (e.g. polypeptides such as antibodies, or polysaccharides) to a solid surface may be achieved using any suitable method available in the art. Where the specific binding partner is a polypeptide, in an embodiment passive adsorption of the polypeptide to the solid surface is preferred. Passive adsorption may be carried out under any suitable conditions, which will be known to persons skilled in the art.

The oligonucleotide may be attached to the solid phase prior to attaching the specific binding partner, or the specific binding partner may be attached to the solid phase before attaching the oligonucleotide. Alternatively, both may be attached at the same time, for example where the specific binding partner is a nucleic acid molecule and a similar method of attachment is used.

### Method of Detection

The present invention provides a method of detection of a target analyte, comprises contacting a sample suspected of containing the target molecule with a solid phase conjugate of the present invention; incubating for sufficient time to allow specific binding of solid phase conjugate to any target molecule present in the sample; and detecting a change which occurs upon binding of the target molecule to the solid phase conjugate. A method of the present invention may be used for a quantitative or qualitative assay.

The present invention provides a method of detecting a target molecule, the method comprising a) providing a solid-phase biomolecule conjugate, wherein the conjugate comprises a solid phase having a surface, wherein the surface has attached thereto i) a specific binding partner for the target molecule, and ii) a population of oligonucleotides which do not bind to the target molecule, and wherein each oligonucleotide is between about 2 to about 200 nucleotides in length;

b) contacting the solid-phase biomolecule conjugate with a sample suspected of containing the target molecule;

c) incubating the sample and the conjugate for sufficient time to allow specific binding of the conjugate to any target molecule present in the sample; and

d) detecting a change which occurs upon binding of any target molecule to the conjugate.

A method of the invention may be used to isolate and/or purify a target molecule present in a sample. The method of the invention relates to increasing the sensitivity of an assay for detection of a target molecule in a sample, by the use of a conjugate.

The population of oligonucleotides as defined herein provide an improved blocking agent compared to conventional blocking agents such as BSA.

In an embodiment, the population of oligonucleotides are provided on the surface of the solid phase over substantially the entire surface which is not bound by the binding partner. They may be provided over the entire surface at uniform or non-uniform density.

In an embodiment, the population of oligonucleotides do not specifically bind to the target. Where the target molecule is a nucleic acid molecule, the oligonucleotides of the population do not bind to the target molecule, or a sequence complementary thereto or an extension or ligation product thereof.

In an embodiment, the oligonucleotides of the population, by virtue of sequence, structure and/or chemical modification, do not bind to or any other molecule present in the sample, either in a specific and/or non-specific manner. The oligonucleotides of the population may be referred to as non-binding. Therefore, in an embodiment of the first aspect, the population of oligonucleotides substantially do not bind to the target molecule, or to any other molecule which may be present in a sample containing or suspected of containing the target molecule. Suitably, the oligonucleotides of the population do not bind specifically to the target molecule or any other molecule which may be present in a sample. In addition, or alternatively, suitably the oligonucleotides of the population do not bind non-specifically to the target molecule or any other molecule which may be present in a sample. The step of contacting a solid phase biomolecule conjugate of the invention with a sample suspected of containing a target molecule may comprise adding the conjugate to a sample, or adding sample to the conjugate. The conjugate may be provided in an assay format, which is then contacted with a sample. For example, the solid phase conjugate may be provided as a dried reagent in a lateral flow assay, which is activated upon wetting or contact with a sample.

The step of incubating the sample and the solid phase conjugate for a suitable length of time for any target molecule to specifically bind to a specific binding partner provided on a solid phase conjugate. This step may take place under conditions which are known to be suitable for hybridisation of nucleic acids, for example where the specific binding partner and target molecule are nucleic acid molecules. Binding conditions for proteins binding to receptors and antibodies, and suitable incubation times, will also be known to persons skilled in the art ort can be determined empirically. Typically, suitable incubation times are known in the art and can be determined by a skilled person. Binding conditions are well known in the art and can readily be optimized for the particular system employed. See, e.g., Sambrook et al., Molecular Cloning: A Laboratory Manual (2nd ed. 1989). Rate of binding may be increased by altering the temperature of the incubation (e.g. freezing or warming), or by increasing the salt concentration (e.g., from 0.I M to I M NaCl).

The detection methods of the invention may be used to determine qualitative or quantitative amounts of a target analyte. Such methods are particularly useful for determining the approximate amount of a target analyte in a sample, which can be used to diagnose certain medical conditions or evaluate the efficacy of a therapy. In one embodiment, the quantity of a target analyte can be determined by establishing a standard curve for the particular molecule by measuring changes in optical signals for samples with a known quantity of target molecule; determining the optical signal change for a test sample; and comparing the optical signal change for the test sample to the values obtained for the standard curve. In some embodiments, determining the quantity of a complex between a solid phase conjugate and target molecule comprises comparing the absorbance ratio and/or reaction rate from a test sample to the absorbance ratio and/or reaction rate from one sample with a known quantity of such a complex, thereby determining the quantity of such a complex in the test sample. The quantitative values obtained from test samples may be compared to pre-determined threshold values, wherein said pre-determined threshold values are indicative of either an abnormal or normal level of the target molecule.

A detectable change that occurs upon hybridization of the solid phase conjugate to a target molecule in the sample may be an optical change (e.g. color change). Various means for measuring optical charges at different wavelengths and acquiring extinction, scattering, or emission spectra are known in the art. Any spectrophotometric or photometric instruments are suitable for use in the disclosed methods. Some non-limiting examples include plate readers, Cobas Fara analyzers, and Piccolo Xpress® and Vetscan analyzers (Abaxis, Inc., Union City, Calif.), optic fiber readers (e.g., LightPath™ S4 (LamdaGen, Menlo Park, Calif.)), SPR instruments (e.g., Biacore instruments available from GE Healthcare), centrifugal analyzers from Olympus, Hitachi etc. The formation of aggregates of the solid phase conjugate or the precipitation of the such aggregates, for example where the solid phase is particulate. The optical changes can be observed with the naked eye or spectroscopically. The formation of aggregates of particles can be observed by electron microscopy or by nephelometry, or by observing or measuring the effects of the aggregation on sample flow for example in a lateral flow assay. The precipitation of aggregated particles can be observed with the naked eye, microscopically or with spectroscopic detection via transmission or reflectance reader methods.

The method of the present invention may be a method of isolating a target molecule, or a method of measuring the amount of a target molecule.

The present invention also relates to the use of a solid-phase biomolecule conjugate for detection of a target molecule in a sample, wherein the conjugate comprises a solid phase having a surface, wherein the surface has attached thereto i) a specific binding partner for the target molecule, and ii) a population of oligonucleotides which are non-specific for the target molecule, and wherein each oligonucleotide is between about 2 to about 200 nucleotides in length.

The present invention relates to the use of a solid-phase biomolecule conjugate for increasing the sensitivity of an assay for detection of a target molecule in a sample, wherein the conjugate comprises a solid phase having a surface, wherein the surface has attached thereto i) a specific binding partner for the target molecule, and ii) a population of oligonucleotides which are non-specific for the target molecule, and wherein each oligonucleotide is between about 2 to about 200 nucleotides in length.

In an embodiment, the population of oligonucleotides are provided on the surface of the solid phase over substantially the entire surface which is not bound by the binding partner. They may be provided over the entire surface at uniform or non-uniform density.

In an embodiment, the population of oligonucleotides do not specifically bind to the target. Where the target molecule is a nucleic acid molecule, the oligonucleotides of the population do not bind to the target molecule, or a sequence complementary thereto or an extension product thereof. Where the specific binding partner is a nucleic acid molecule, the oligonucleotides of the population do not bind to a substantially identical sequence, a sequence complementary to the binding partner nucleic acid molecule, an extension product of the binding partner nucleic acid molecule, or a ligation product of the binding partner nucleic acid molecule.

In an embodiment, the oligonucleotides of the population, by virtue of sequence, structure and/or chemical modification, do not bind to or any other molecule present in the sample, either in a specific and/or non-specific manner. The oligonucleotides of the population may be referred to as non-binding. Therefore, in an embodiment the population of oligonucleotides substantially do not bind to the target molecule, or to any other molecule which may be present in a sample containing or suspected of containing the target molecule. Suitably, the oligonucleotides of the population do not bind specifically to the target molecule or any other molecule which may be present in a sample. In addition, or alternatively, suitably the oligonucleotides of the population do not bind non-specifically to the target molecule or any other molecule which may be present in a sample.

### Kit

The invention further provides a kit for performing the assays for detecting or quantitating analytes. The kit comprises a container comprising a solid phase biomolecule conjugate of the invention. The kit may also contain other reagents and items useful for performing the assays. The reagents may include controls, standards, PCR reagents, hybridization reagents, buffers, etc. Other items which be provided as part of the kit include reaction devices (e. g, test tubes, microtiter plates, syringes, pipettes, cuvettes, containers, etc. The kits of the invention may also include instructions for using the device to detect an analyte in a test sample, devices or tools for collecting biological samples, and/or extraction buffers for obtaining samples from solid materials, such as soil, food, and biological tissues.

The articles “a” and “an” are used herein to refer to one or to more than one (i.e. to at least one) of the grammatical object of the article. By way of example, “an antibody” means one antibody or more than one antibody. The term “at least” is used to indicate that optionally one or more further objects may be present. By way of example, an array comprising at least two discrete areas may optionally comprise two or more discrete test areas.

The invention is not restricted to the details of any forgoing embodiments.

Throughout the description and claims of this specification, the words “comprise” and “contain” and variations of them mean “including but not limited to”, and they are not intended to (and do not) exclude other components. Throughout the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular embodiment of the invention are to be understood to be applicable to any other embodiment described herein unless incompatible therewith. All of the features disclosed in this specification (including any accompanying claims and drawings) may be combined in any combination, except combinations where at least some of such features and/or steps are mutually exclusive. The invention extends to any novel one, or any novel combination, of the features disclosed in this specification (including any accompanying claims and drawings).

### EXAMPLES

The following specific examples are merely illustrative and accordingly this invention may be embodied in many forms and is therefore not limited to the following methods and materials. It is thought that one skilled in the art can make use of the present invention to the full extent based on the detailed description and specific example herein.

### Example 1

In this example, a conjugate embodiment was prepared for the detection of Brain Natriuretic Peptide (BNP). 40 nm gold nanoparticles supplied by BBI Solutions (EM.GC40), diluted in purified water (Resistivity ≥18MΩ) to an optical density (OD) of 1.0 at A_{520 nm} (measured using a Hitachi UV/VIS spectrophotometer, model U2800A). Monoclonal anti BNP IgG antibody (Purchased from HyTest Ltd, product code: 4BN P2 Mab 24C5) was attached non-covalently to 40 nm gold nanoparticles using any known method for linking proteins to gold nanoparticles. In this embodiment, the monoclonal anti BNP antibody was non-covalently attached to the 40 nm gold nanoparticles at a pH of 6.0, pH adjusted with 0.1% HCl. After pH adjustment, 1 ml of OD 1 antibody-gold nanoparticle mixture was removed and placed in a 1.5 ml low bind Eppendorf tube (Eppendorf, product code: 022431021).

A 10% (v/v) solution of Tween 80 (Purchased from VWR, product code:28830.291) was made up in purified water (Resistivity ≥18MΩ) and 4 μl of 10% Tween 80 solution was added to the 1 ml OD1 antibody-gold nanoparticle mixture. The antibody-gold nanoparticle mixture was incubated with the 10% Tween 80 solution at room temperature, typically 18-24° C., with constant mixing at low speed for 30 minutes. Following the 30 minute incubation, 100 μl of a phosphate buffered saline buffer (PBS) (0.1M Na₂ HPO₄, 0.03M KH₂O₄ P, 1.23M NaCl, pH7.4) was added to the 1 ml OD 1 antibody-gold nanoparticle mixture. This was added dropwise, with inversion after each drop. Immediately after the PBS addition, the oligonucleotide was added, 10 μl of 100 μM oligonucleotide (diluted to 100 μM concentration in DNase/RNase free water, purchased from Sigma, product code: W4502). In this embodiment, a 10 base poly T sequence with thiol modification was used (5′ SH-TTT-TTT-TTT-T 3′) (purchased from Eurogentec).

The solution was placed into a 50° C. water-bath and left to incubate with the oligonucleotide for 1 hr 20 mins. The solution was inverted four times during the incubation to allow sufficient mixing. After the incubation, the conjugate was centrifuged at 4720 rcf (relative centrifugal force) for 15 minutes. The supernatant was removed and the pellet re-suspended in 2 mM Borax pH 9.0 (2 mM B₄ Na₂ O₇. 10H₂O, 0.095% NaN₃) to give a final conjugate OD of 10 at A_{520 nm}.

### Testing of Conjugate

A control conjugate was manufactured using the Monoclonal anti BNP IgG antibody (Purchased from HyTest Ltd, product code: 4BNP2 Mab 24C5), which was non-covalently attached to the 40 nm gold nanoparticles at a pH of 6.0, and blocked with an excess of BSA rather than oligonucleotide.

An antigen dilution buffer of Phosphate Buffered Saline, pH7.2 was produced (0.01M Na₂ HPO₄, 0.003M K H₂ PO₄, 0.123M NaCl), and used to dilute the BNP-32 antigen (purchased from Bachem, catalogue number H-9060.0500), from 500 μg/ml stock concentration, to the working concentrations required.

A sample panel was prepared from the BNP-32 stock antigen to give serial dilutions of antigen of 0.01 ng/ml, 0.02 ng/ml, 0.04 ng/ml, 0.06 ng/ml, 0.08 ng/ml, 0.1 ng/ml, 0.12 ng/ml, 0.14 ng/ml, 0.16 ng/ml, 0.18 ng/ml, 0.2 ng/ml, 0.4 ng/ml, 0.8 ng/ml, 1.6 ng/ml, 3.2 ng/ml. A buffer only control (PBS pH7.2) was used as the negative control.

Both the oligonucleotide-blocked anti-BNP conjugate and the BSA-blocked anti-BNP conjugates were diluted to OD1 concentrations (measured at A_{520 nm}), using Phosphate Buffered Saline 1% Tween20(v/v) pH7.2 (0.01M Na₂ HPO₄, 0.003M K H₂ PO₄, 0.123M NaCl, 1% Tween20).

Lateral flow half dipsticks were manufactured using a nitrocellulose membrane supported by an absorbent upper wick. Millipore Hi-Flow Plus HF135 Membrane Cards 60 mm×301 mm, was used as the nitrocellulose membrane and Ahlstrom 222 as the upper wick (21 mm width reeled material used to laminate Millipore cards). Half dipsticks were then produced by cutting the cards to 4 mm width dipsticks. No sample or conjugate pad was used during the half dipstick testing, the strips were placed upright into wells of a 96 well-plate (purchased from Greiner, bio-one 96 well Microplate PS, F-bottom, clear, REF655101). The Anti-BNP capture antibody (purchased from HyTest Ltd, catalogue number 4BNP2 50E1 Anti-BNP) was immobilised onto the HF135 Nitrocellulose at 1 mg/ml concentration, using a dilution buffer of PBS pH7.2. (0.01M Na₂ HPO₄, 0.003M K H₂ PO₄, 0.123M NaCl). 20 μl of OD1 gold-antibody conjugate diluted in PBS 1% Tween20 pH7.2, and 20 μl BNP-32 antigen diluted in PBS pH7.2 were pipetted into a well of the 96 well plate, left to incubate for 2 minutes, before the plate was shaken to mix, and a lateral flow dipstick was added. Three replicates were run for each BNP-32 antigen concentration tested, with all strips run until no sample and conjugate mix remained in the well. Strips were then transferred into a separate well containing 20 μl PBS 1% Tween20 pH7.2 buffer, to clear any excess and unbound conjugate from the strips. Strips were read following the running of the buffer-only wells using a Camag TLC Scanner 3, measuring the reflectance at 520 nm.

Mean signal intensities were calculated and plotted for each of the concentrations tested, with the standard deviation and standard error also calculated. 2× Standard Error was used to produce error bars displayed in FIG. 2.

| | | Camag Units | | | |
|---|---|---|---|---|---|
| | Concen- | | | | 2 × |
| | tration of | Test | Mean | Stan- | Stan- |
| Conjugate | BNP in | line | Test | dard | dard |
| condition | ng/ml | signal | line | Deviation | Error |
| BSA-blocked | 0.00 | 15.50 | 16.80 | 2.76 | 2.47 |
| control | | 15.30 | | | |
| conjugate | | 16.10 | | | |
| | | 15.40 | | | |
| | | 21.70 | | | |
| | 0.01 | 15.70 | 15.42 | 0.76 | 0.68 |
| | | 15.20 | | | |
| | | 14.90 | | | |
| | | 16.60 | | | |
| | | 14.70 | | | |
| | 0.02 | 15.30 | 16.22 | 1.00 | 0.90 |
| | | 16.70 | | | |
| | | 17.60 | | | |
| | | 16.30 | | | |
| | | 15.20 | | | |
| | 0.04 | 16.40 | 16.88 | 1.79 | 1.60 |
| | | 19.10 | | | |
| | | 15.30 | | | |
| | | 15.20 | | | |
| | | 18.40 | | | |
| | 0.06 | 16.10 | 15.06 | 1.25 | 1.11 |
| | | 14.30 | | | |
| | | 14.00 | | | |
| | | 16.70 | | | |
| | | 14.20 | | | |
| | 0.08 | 14.70 | 17.42 | 2.46 | 2.20 |
| | | 16.30 | | | |
| | | 19.50 | | | |
| | | 20.50 | | | |
| | | 16.10 | | | |
| | 0.10 | 16.20 | 15.30 | 1.05 | 0.94 |
| | | 13.70 | | | |
| | | 14.80 | | | |
| | | 16.10 | | | |
| | | 15.70 | | | |
| | 0.12 | 16.10 | 16.00 | 0.82 | 0.73 |
| | | 16.40 | | | |
| | | 15.90 | | | |
| | | 16.90 | | | |
| | | 14.70 | | | |
| | 0.14 | 15.20 | 14.58 | 0.59 | 0.53 |
| | | 13.70 | | | |
| | | 14.80 | | | |
| | | 14.90 | | | |
| | | 14.30 | | | |
| | 0.16 | 16.20 | 15.76 | 1.63 | 1.46 |
| | | 18.40 | | | |
| | | 15.00 | | | |
| | | 14.30 | | | |
| | | 14.90 | | | |
| | 0.18 | 16.40 | 17.10 | 1.26 | 1.13 |
| | | 17.70 | | | |
| | | 18.70 | | | |
| | | 17.30 | | | |
| | | 15.40 | | | |
| | 0.20 | 16.50 | 17.22 | 0.79 | 0.71 |
| | | 16.90 | | | |
| | | 17.80 | | | |
| | | 18.30 | | | |
| | | 16.60 | | | |
| | 0.40 | 20.70 | 18.82 | 2.50 | 2.23 |
| | | 18.00 | | | |
| | | 21.90 | | | |
| | | 17.90 | | | |
| | | 15.60 | | | |
| | 0.80 | 23.80 | 29.26 | 5.80 | 5.19 |
| | | 22.50 | | | |
| | | 35.40 | | | |
| | | 33.50 | | | |
| | | 31.10 | | | |
| | 1.60 | 79.30 | 108.88 | 25.07 | 22.43 |
| | | 99.30 | | | |
| | | 129.70 | | | |
| | | 96.30 | | | |
| | | 139.80 | | | |
| | 3.20 | 284.60 | 286.86 | 31.19 | 27.89 |
| | | 323.00 | | | |
| | | 245.50 | | | |
| | | 311.10 | | | |
| | | 270.10 | | | |

TABLE 2
Signal intensities produced by oligo-blocked conjugate
during anti-BNP conjugate comparison

| | | Camag Units | | | |
|---|---|---|---|---|---|
| | Concen- | | | | 2 × |
| | tration of | Test | Mean | Stan- | Stan- |
| Conjugate | BNP in | line | Test | dard | dard |
| condition | ng/ml | signal | line | Deviation | Error |
| Oligonucleotide- | 0.00 | 19.60 | 19.60 | 0.45 | 0.40 |
| blocked | | 20.00 | | | |
| conjugate | | 20.10 | | | |
| | | 19.10 | | | |
| | | 19.20 | | | |
| | 0.01 | 20.90 | 20.30 | 2.00 | 1.79 |
| | | 22.70 | | | |
| | | 20.00 | | | |
| | | 20.70 | | | |
| | | 17.20 | | | |
| | 0.02 | 22.00 | 21.76 | 1.43 | 1.28 |
| | | 21.80 | | | |
| | | 20.00 | | | |
| | | 23.90 | | | |
| | | 21.10 | | | |
| | 0.04 | 21.20 | 21.20 | 0.64 | 0.57 |
| | | 21.50 | | | |
| | | 20.10 | | | |
| | | 21.50 | | | |
| | | 21.70 | | | |
| | 0.06 | 16.90 | 19.68 | 3.47 | 3.10 |
| | | 23.40 | | | |
| | | 22.50 | | | |
| | | 20.20 | | | |
| | | 15.40 | | | |
| | 0.08 | 20.30 | 21.50 | 1.28 | 1.15 |
| | | 22.90 | | | |
| | | 22.60 | | | |
| | | 20.10 | | | |
| | | 21.60 | | | |
| | 0.10 | 22.30 | 24.30 | 1.82 | 1.63 |
| | | 25.20 | | | |
| | | 22.60 | | | |
| | | 26.60 | | | |
| | | 24.80 | | | |
| | 0.12 | 27.70 | 25.18 | 4.50 | 4.02 |
| | | 19.10 | | | |
| | | 21.70 | | | |
| | | 29.30 | | | |
| | | 28.10 | | | |
| | 0.14 | 20.30 | 25.12 | 4.81 | 4.30 |
| | | 28.50 | | | |
| | | 25.70 | | | |
| | | 20.20 | | | |
| | | 30.90 | | | |
| | 0.16 | 31.10 | 29.88 | 0.90 | 0.81 |
| | | 30.10 | | | |
| | | 28.60 | | | |
| | | 30.00 | | | |
| | | 29.60 | | | |
| | 0.18 | 38.00 | 35.24 | 3.25 | 2.91 |
| | | 35.10 | | | |
| | | 29.90 | | | |
| | | 37.70 | | | |
| | | 35.50 | | | |
| | 0.20 | 34.40 | 32.56 | 4.34 | 3.88 |
| | | 35.30 | | | |
| | | 33.40 | | | |
| | | 24.90 | | | |
| | | 34.80 | | | |
| | 0.40 | 40.70 | 53.24 | 7.91 | 7.07 |
| | | 51.00 | | | |
| | | 55.70 | | | |
| | | 57.70 | | | |
| | | 61.10 | | | |
| | 0.80 | 110.40 | 108.48 | 19.09 | 17.07 |
| | | 82.50 | | | |
| | | 96.90 | | | |
| | | 128.50 | | | |
| | | 124.10 | | | |
| | 1.60 | 234.20 | 251.80 | 29.82 | 26.68 |
| | | 229.00 | | | |
| | | 263.20 | | | |
| | | 233.30 | | | |
| | | 299.30 | | | |
| | 3.20 | 492.30 | 451.44 | 34.48 | 30.82 |
| | | 484.10 | | | |
| | | 420.20 | | | |
| | | 421.80 | | | |
| | | 438.80 | | | |

TABLE 3
Mean signal intensities produced during
anti-BNP conjugate comparison

| Concentration | BSA block | | Oligonucleotide block | |
|---|---|---|---|---|
| in ng/ml | T mean | T 2 × SE | T mean | T 2 × SE |
| Negative | 16.80 | 2.47 | 19.60 | 0.40 |
| 0.01 | 15.42 | 0.68 | 20.30 | 1.79 |
| 0.02 | 16.22 | 0.90 | 21.76 | 1.28 |
| 0.04 | 16.88 | 1.60 | 21.20 | 0.57 |
| 0.06 | 15.06 | 1.11 | 19.68 | 3.10 |
| 0.08 | 17.42 | 2.20 | 21.50 | 1.15 |
| 0.10 | 15.30 | 0.94 | 24.30 | 1.63 |
| 0.12 | 16.00 | 0.73 | 25.18 | 4.02 |
| 0.14 | 14.58 | 0.53 | 25.12 | 4.30 |
| 0.16 | 15.76 | 1.46 | 29.88 | 0.81 |
| 0.18 | 17.10 | 1.13 | 35.24 | 2.91 |
| 0.20 | 17.22 | 0.71 | 32.56 | 3.88 |
| 0.40 | 18.82 | 2.23 | 53.24 | 7.07 |
| 0.80 | 29.26 | 5.19 | 108.48 | 17.07 |
| 1.60 | 108.88 | 22.43 | 251.80 | 26.68 |
| 3.20 | 286.86 | 27.89 | 451.44 | 30.82 |

### CONCLUSIONS

The oligonucleotide blocked anti-BNP conjugate achieved a lower limit of detection than the BSA-blocked control conjugates. A limit of detection of 0.08 ng/ml, or 80 μg/ml, was achieved with the oligonucleotide blocker in comparison to the BSA blocked conjugates which achieved a limit of detection of 0.8 ng/ml BNP-32 antigen.

The oligonucleotide blocked conjugate enabled the visual signal intensity at each concentration of BNP-32 tested to be discernably higher than those produced by the control conjugates, with the biggest contrast in signals observed at the 0.8 ng/ml concentration.

The increase in sensitivity achieved by the oligonucleotide-blocking in this assay system has enabled the assay to discriminate between BNP-32 concentrations within the clinically-relevant range. This demonstrates the advantages the improvements in assay sensitivity may deliver.

### Example 2

The blocking of the nano-particle using populations of oligonucleotides has been demonstrated in Example 1 using single stranded DNA sequences, which are non-specific for the target molecule and show improved sensitivity in a detection assay compared to conventional blocking agents such as BSA. The experiments below further demonstrate that the oligonucleotides on the particle do not conduct any hybridisation interaction in the process. The oligonucleotides can be made up of single stranded oligonucleotides non-specific to the target but capable of hybridisation, single stranded oligonucleotides not capable of hybridisation, or hybridised oligonucleotides in a duplex.

### Method of Conjugation

In this example, a conjugate embodiment was prepared for the detection of Human Chorionic Gonadotropin (hCG). 40 nm gold nanoparticles supplied by BBI Solutions (EM.GC40), diluted in purified water (Resistivity ≥18MΩ) to an optical density (OD) of 1.0 at A_{520 nm} (measured using a Hitachi UV/VIS spectrophotometer, model U2800A). Monoclonal anti hCG IgG antibody (Purchased from Medix Biochemica, product code: 100006 Anti-HCG 5008 SP-5) was attached non-covalently to 40 nm gold nanoparticles using any known method for linking proteins to gold nanoparticles. The monoclonal anti hCG antibody was non-covalently attached to the 40 nm gold nanoparticles at a pH of 7.0, pH adjusted with 0.1% HCl. After pH adjustment, 1 ml of OD 1 antibody-gold nanoparticle mixture was removed and placed in a 1.5 ml low bind Eppendorf tube (Eppendorf, product code: 022431021).

A 10% (v/v) solution of Tween 80 (Purchased from VWR, product code:28830.291) was made up in purified water (Resistivity ≥18MΩ) and 4 μl of 10% Tween 80 solution was added to the 1 ml OD1 antibody-gold nanoparticle mixture. The antibody-gold nanoparticle mixture was incubated with the 10% Tween 80 solution at room temperature, typically 18-24° C., with constant mixing at low speed for 30 minutes. Following the 30 minute incubation, 100 μl of a phosphate buffered saline buffer (PBS) (0.1M Na₂ HPO₄, 0.03M KH₂O₄ P, 1.23M NaCl, pH7.4) was added to the 1 ml OD 1 antibody-gold nanoparticle mixture. This was added dropwise, with inversion after each drop. Immediately after the PBS addition, the oligonucleotide was added, 10 μl of 100 μM oligonucleotide (diluted to 100 μM concentration in DNase/RNase free water, purchased from Sigma, product code: W4502). In this embodiment, a 10 base poly T sequence with thiol modification was used (5′ SH-TTT-TTT-TTT-T 3′) (purchased from Eurogentec).

The solution was placed into a 50° C. water-bath and left to incubate with the oligonucleotide for 1 hr 20 mins. The solution was inverted four times during the incubation to allow sufficient mixing. After the incubation, the conjugate was centrifuged at 4720 relative centrifugal force for 15 minutes. The supernatant was removed and the pellet re-suspended in 2 mM Borax pH 9.0 (2 mM B₄ Na₂ O₇. 10H₂O, 0.095% NaN₃) to give a final conjugate OD of 10 at A_{520 nm}.

### Hybridisation of Oligonucleotide

100 μl of OD10 conjugate was pipette into 2 Eppendorf's. 1 Eppendorf had 50 μl of 100 mM unmodified poly A oligonucleotide (5′ AAA-AAA-AAA-A 3′) added to the conjugate, and the volume was made up to 1050 μl with PBS 1% Tween20 pH7.2 buffer. The other Eppendorf was made up to the 1.05 ml with 1 ml of PBS 1% Tween20 pH7.2, and acted as the control conjugate.

The conjugates were mixed for 1 hour at room temperature, allowing for the poly A oligonucleotide to hybridise with the poly T oligonucleotide previously attached when blocking the anti-hCG conjugate. The inclusion of the control conjugate allowed for a comparison between single stranded poly T oligonucleotide and duplexed oligonucleotides, when used to block the unbound sites on gold particles.

### Testing of Conjugates

Lateral flow half dipsticks were manufactured using a nitrocellulose membrane supported by an absorbent upper wick. Millipore Hi-Flow Plus HF135 Membrane Cards 60 mm×301 mm, was used as the nitrocellulose membrane and Ahlstrom 222 as the upper wick (21 mm width reeled material used to laminate Millipore cards). Half dipsticks were then produced by cutting the cards to 4 mm width dipsticks. No sample or conjugate pad was used during the half dipstick testing, the strips were placed upright into wells of a 96 well-plate (purchased from Greiner, bio-one 96 well Microplate PS, F-bottom, clear, REF655101). The Anti-hCG capture antibody (purchased from Medix Biochemica, Anti-hCG Alpha subunit 6601/100066) was immobilised onto the HF135 Nitrocellulose at 1 mg/ml concentration, using a dilution buffer of PBS pH7.2. (0.01M Na₂ HPO₄, 0.003M K H₂ PO₄, 0.123M NaCl). A control—line of 10 base poly A oligonucleotide had also been previously immobilised onto the Nitrocellulose, so allowing for a hybridisation control during the running of the strips.

20 μl of the OD1 gold-antibody conjugates previously diluted in PBS 1% Tween20 pH7.2, and 20 μl hCG antigen diluted in PBS pH7.2 were pipetted into a well of the 96 well plate, left to incubate for 2 minutes, before the plate was shaken to mix, and a lateral flow dipstick was added. Five replicates were run for each hCG antigen concentration tested, with all strips run until no sample and conjugate mix remained in the well. Strips were then transferred into a separate well containing 20 μl PBS 1% Tween20 pH7.2 buffer, to clear any excess and unbound conjugate from the strips. Strips were read following the running of the buffer-only wells using a Camag TLC Scanner 3, measuring the reflectance at 520 nm.

Mean signal intensities were calculated and plotted for each of the concentrations tested, with the standard deviation and standard error also calculated. 2×Standard Error was used to produce error bars displayed in the graph.

### Results

TABLE 1
Camag raw data readings

| | Concen- | Test line | | | |
|---|---|---|---|---|---|
| | tration of | signal in | Mean Test line | | |
| Conjugate | hCG in | Camag | signal in | | 2 × |
| conditions | IU/L | Units | Camag Units | s.d | SE |
| Control | 0 | 7.3 | 10.3 | 5.3 | 4.7 |
| conjugate - | | 19.6 | | | |
| Poly T 10 | | 9.8 | | | |
| base oligo | | 7.4 | | | |
| block | | 7.6 | | | |
| | 1 | 11.7 | 12.2 | 0.9 | 0.8 |
| | | 13.1 | | | |
| | | 12.9 | | | |
| | | 12.5 | | | |
| | | 10.9 | | | |
| | 10 | 46.6 | 46.7 | 1.2 | 1.0 |
| | | 45.4 | | | |
| | | 47.7 | | | |
| | | 48.1 | | | |
| | | 45.9 | | | |
| | 100 | 289.8 | 299.7 | 9.8 | 8.8 |
| | | 303.7 | | | |
| | | 288.6 | | | |
| | | 307.2 | | | |
| | | 309.2 | | | |
| Hybridised poly | 0 | 10.9 | 10.0 | 2.0 | 1.8 |
| T - poly A | | 11.4 | | | |
| oligonucleotide | | 11.5 | | | |
| block | | 6.8 | | | |
| | | 9.3 | | | |
| | 1 | 12.4 | 12.8 | 2.2 | 2.0 |
| | | 11.8 | | | |
| | | 10.3 | | | |
| | | 13.1 | | | |
| | | 16.2 | | | |
| | 10 | 45.9 | 47.9 | 1.5 | 1.3 |
| | | 49.4 | | | |
| | | 48.9 | | | |
| | | 48.6 | | | |
| | | 46.9 | | | |
| | 100 | 290.2 | 301.8 | 10.3 | 9.2 |
| | | 291.8 | | | |
| | | 311.5 | | | |
| | | 311.4 | | | |
| | | 303.9 | | | |

### CONCLUSIONS

The Anti-hCG conjugate with unhybridised poly T oligonucleotide blocker, produced a strong and clear visible signal on lateral flow strips at the poly A immobilised oligonucleotide line. Binding at this line shows the poly T on the gold particles are able to hybridise with the 10 base poly A on the Nitrocellulose.

The Anti-hCG conjugate with hybridised poly T—poly A oligonucleotide blocker did not demonstrate any binding with the poly A line on the Nitrocellulose, as shown by the lack of signals at the control line in all images. The lack of signals at the poly A line is due to the poly T blocker hybridising to the poly A oligonucleotide which had been incubated with the conjugate. The results at the control line also show that the blockers on the gold particles in this conjugate are incapable of further hybridisation.

Test line signals of both conjugates remain comparable across the sample concentrations tested. This can be observed from the images and graph included on the data.

The test-line responses demonstrate that both conjugates, whether using unhybridised poly T blocker capable of hybridisation to poly A oligonucleotides, or hybridised poly T—poly A oligonucleotide blocker incapable of further hybridisation, exhibit comparable test performance within the lateral flow format.

These results prove that the blocking of the gold particles and subsequent specific interaction by the antibodies on the particles, have independent functions. The results also show that the oligonucleotides function within the conjugates is blocking un-bound sites on the gold particles, and the oligonucleotides do not participate in any way in the specific interaction. Oligonucleotides not capable of hybridisation show similar results to single stranded oligonucleotides as improved blocking agents.

1. A solid phase biomolecule conjugate for specific binding to a target molecule, the conjugate comprising a solid phase comprising a surface, wherein the surface has bound thereto i) a binding partner specific for the target molecule, and ii) a population of oligonucleotides which do not bind to the target molecule, wherein each oligonucleotide is between 2 to 200 nucleotides in length.

2. A solid phase biomolecule conjugate according to claim 1 wherein the population of oligonucleotides are provided on the surface of the solid phase over substantially the entire surface which is not bound by the binding partner.

3. A solid phase biomolecule conjugate according to claim 1 wherein the oligonucleotides of the population do not bind to or any other molecule present in the sample, either in a specific or non-specific manner, and preferably do not bind to the specific binding partner.

4-5. (canceled)

6. A solid phase biomolecule conjugate according to claim 1 wherein the oligonucleotides of the population each independently comprises or consists of a sequence selected from a poly(A), poly(T), poly(C) or poly(G) sequence.

7. A solid phase biomolecule conjugate according to claim 1 wherein each or a oligonucleotide of the population is a 10 base poly T sequence with a thiol modification or is a poly T sequence/poly A sequence duplex.

8. A solid phase biomolecule conjugate according to claim 1 wherein the oligonucleotides of the population are each independently a double stranded molecule; or comprise a blocking group such as a chemical species.

9. A solid phase biomolecule conjugate according to claim 1 wherein the solid phase is a chip, a particle, a well, a cuvette, a column, a membrane, an array, quantum dots or a bead.

10-12. (canceled)

13. A solid phase biomolecule conjugate according to claim 1 wherein the surface comprises gold or the particle is a gold particle.

14. A solid phase biomolecule conjugate according to claim 1 wherein a specific binding partner is either a biological molecule selected from a nucleic acid molecule, a polypeptide comprising an antibody, an antibody fragment or other polypeptide, a hapten or a polysaccharide; or non-biological selected from a small molecule and a drug.

15-21. (canceled)

22. A solid phase biomolecule conjugate according to claim 1, for detecting a target molecule, wherein the target molecule is either a biological molecule selected from a nucleic acid molecule, a polypeptide, a hapten, or a polysaccharide, a biomolecule or combinations thereof such as nucleic acid-protein complexes, microorganisms, and viruses; or a non-biological molecule selected from a small molecule or drug, or a metal ion.

23-28. (canceled)

29. A solid phase biomolecule conjugate according to claim 1 wherein the oligonucleotides of the population are each independently 2 nucleotides to 200 nucleotides in length.

30. A solid phase biomolecule conjugate according to claim 1 wherein a blocking agent is not present on the surface of the solid-phase biomolecule conjugate.

31-32. (canceled)

33. A method of detecting a target molecule, the method comprising
a) providing a solid-phase biomolecule conjugate, wherein the conjugate comprises a solid phase having a surface, wherein the surface has attached thereto i) a specific binding partner for the target molecule, and ii) a population of oligonucleotides which do not bind the target molecule, and wherein each oligonucleotide is between 2 to 200 nucleotides in length;
b) contacting the solid-phase biomolecule conjugate with a sample suspected of containing the target molecule;
c) incubating the sample and the conjugate for sufficient time to allow specific binding of the conjugate to any target molecule present in the sample; and
d) detecting a change which occurs upon binding of any target molecule to the conjugate.

34. A method according to claim 33 wherein the oligonucleotides of the population each independently comprise 2 nucleotides to 200 nucleotides in length.

35. (canceled)

36. A method for making a conjugate as defined in claim 1, wherein the method comprises i) providing a solid phase of the conjugate comprising a surface comprising gold; ii) attaching a specific binding partner for a target molecule to the surface, wherein the binding partner is either a biological molecule selected from a nucleic acid molecule, a polypeptide, a hapten, or a polysaccharide, or a non-biological selected from a small molecule and a drug; and iii) attaching a population of oligonucleotides to the surface which do not bind to the target molecule, and wherein each oligonucleotide is between 2 to 200 nucleotides in length.

37. (canceled)

38. A method according to claim 36 wherein each or a oligonucleotide is a 10 base poly T sequence with a thiol modification.

39. (canceled)

40. A method according to claim 36, wherein the step of attaching in step ii), step iii), or both steps ii) and iii) is independently selected from conjugation chemistries comprising 1 Ethyl-3-[3-dimethylaminopropyl]carbodiimide hydrochloride (EDC), sulfo-NHS coupling, hydrophobic binding or thioether chemistry, or is via a functional group associated with the solid surface or the oligonucleotide or specific binding partner, respectively.

41. A method according to claim 36 wherein step ii) comprises attaching a specific binding partner indirectly to the solid phase via a larger carrier molecule or protein or attaching a specific binding partner to the solid phase surface by passive adsorption.

42. (canceled)

43-46. (canceled)

47. A method according to claim 36 wherein step ii) comprises providing suitable conditions to allow a covalent or non-covalent bond to form between a reactive group provided on a oligonucleotide and a gold surface of the conjugate.

48. A method according to claim 36 wherein the bond is covalent, and step iii) comprises incubating the solid phase with the oligonucleotide population in the presence of salt and non-ionic surfactant.

49-51. (canceled)
